(19) United States
(12) Patent Application Publication
(10) Pub. No.: US 2009/0263497 A1
(43) Pub. Date: Oct. 22, 2009

(54) PRODUCTION METHOD FOR CALCIUM PHOSPHATE NANO-PARTICLES WITH HIGH PURITY AND THEIR USE

(75) Inventors: Jose Carlos Brito Lopes, Porto (PT); Madalena Maria Gomes De Queiroz Dias, Porto (PT); Viviana Manueal Tenedorio Matos Da Silva, Tinto (PT); Paulo Alexandre Quadros De Oliviera E Santos, Salreu (PT); Fernando Jorge Mendes Monteiro, Porto (PT); Paulo Jorge Da Cunha Gomes, Leca da Palmeira (PT); Alis Yovana Pataquiva Mateus, S.Mamede Infesta (PT)

Correspondence Address: SUGHRUE MION, PLLC, 2100 PENNSYLVANIA AVENUE, N.W., SUITE 800, WASHINGTON, DC, 20037, US

(73) Assignees: FLUDINOVA, ENGENHARIA DE FLUIDOS, S.A., Moreira Da Maia (PT); INSTITUTO NACIONAL DE ENGENHARIA BIOMEDICA, Porto (PT)

(21) Appl. No.: 12/159,696

(22) PCT Filed: Jul. 16, 2007
(86) PCT No.: PCT/PT07/00031

§ 371 Date: Apr. 27, 2009

(30) Foreign Application Priority Data
Jul. 14, 2006 (PT) 103528

(51) Int. Cl. C01B 25/32; A61K 33/42; A61K 47/02

(52) U.S. Cl. 424/602; 423/311; 423/306; 423/301; 514/769

(57) ABSTRACT

The present invention provides a continuous process for producing calcium phosphate nanoparticles in a network mixer or static mixer reactor, fed by a calcium solution, a phosphorous solution and an alkaline solution and, optionally, one solvent or dispersing agent. The proposed process enables the micromixing control, which is essential to form nanometric structures, but it is also a determining factor in the crystals purity, crystallinity and morphology. The reactants distribution scheme at the inlet of the reactor and along the reactor, performed continuously or varying in time, is also a crucial factor to programme the pH of the reactant media along the reactor The calcium phosphate nanoparticles suspension that exits the reactor can be submitted to further aging, ultra-sounds, separation, drying, sintering and milling processes. Some calcium phosphates are considered biomaterials, used as: food additives and nutritional supplements; bone graft for bone replacement, growth and repair; biocements and coating of metallic implant. Some of the most recent applications include their use in cosmetics, toothpaste and in esthetical treatments for diminishing wrinkles by stimulating conjunctive tissue formation.

### FIELD OF THE INVENTION

The present invention provides a continuous process for producing calcium phosphate nanoparticles in a network mixer or static mixer reactor, fed by a calcium solution, a phosphorous solution and an alkaline solution and, optionally, one solvent or surfactant agent, being therefore related with the chemical industry synthesis domain.

### SUMMARY OF THE INVENTION

The present invention provides a continuous process for producing calcium phosphate nanoparticles in a network mixer or static mixer reactor, fed by a calcium solution, a phosphorous solution and an alkaline solution and, optionally, one solvent or surfactant agent.

The proposed process enables the micromixing control, which is essential to form nanometric structures, but it is also a determining factor in the crystals purity, crystallinity and morphology. The reactants distribution scheme at the inlet of the reactor and along the reactor, performed continuously or varying in time, is also a crucial factor to programme the pH of the reactant media along the reactor. The calcium phosphate nanoparticles suspension that exits the reactor can be submitted to further aging, ultra-sounds, separation, drying, sintering and milling processes.

### BACKGROUND OF THE INVENTION

Calcium Phosphates (CP's) are inorganic compounds constituted by Ca²⁺ and PO₄³⁻ ions at different stoichiometric amounts, see Table 1; furthermore the substitution of some ions of the crystallographic structure by ions such as F⁻, Na⁺, K⁺, Mg²⁺ and CO₃²⁺ can provide different properties to the substituted calcium phosphates. Some calcium phosphates are considered as biomaterials and, therefore, are used in several food, biomedical and pharmaceutical applications; and more recently are also used in the cosmetic industry.

TABLE 1
Calcium Phosphates: name, short-name and chemical formula

| Name | Short-name | Chemical Formula |
|---|---|---|
| Monocalcium Phosphate | MCPM | Ca(H₂PO₄)₂ × H₂O |
| Monohydrate | | |
| Dicalcium Phosphate | DCPD | CaHPO₄ × 2H₂O |
| Dihydrate | | |
| Dicalcium Phosphate | DCPA | CaHPO₄ |
| Anhydrous | | |
| Octacalcium Phosphate | OCP | Ca₈H₂(PO₄)₆ × 5H₂O |
| Amorphous Calcium | ACP | Ca₃(PO₄)₂ |
| Phosphate | | |
| α-Tricalcium Phosphate | α-TCP | α-Ca₃(PO₄)₂ |
| β-TriCalcium Phosphate | β-TCP | β-Ca₃(PO₄)₂ |
| Hydroxyapatite | HAp | Ca₁₀(PO₄)₆(OH)₂ |
| TetraCalcium Phosphate | TTCP | Ca₄(PO₄)₂O |

The most important biocompatible calcium phosphates are the Hydroxyapatite (HAp) that is considered a bioactive ceramic, and the β-Tricalcium Phosphate (β-TCP) that is a bioresorbable ceramic. HAp is the main mineral component of the human bone, and its bioactivity makes HAp thermodynamically stable in physiologic environments, promoting at the surface of the bone implant a strong biological and chemical reaction with the surrounding tissue. β-TCP, like most of all CP's (excluding HAp) is considered as a bioresorbable material due to its dissolution when exposed to physiological environments making possible the natural bone replacement. The reabsorbing rate is directly proportional to the CP solubility, which is also affected by the pH. Generally, CP's can be ordered by their decreasing solubility as follows:

DCPD>DCPA>ACP>TTCP>α-TCP>OCP>β-TCP>HAp.

In state-of-the-art, it can be found several methods for CP's production, mainly CP's can be prepared wet chemical reactions and by solid-state reactions. The wet chemical process have different routs as chemical precipitation, hydrothermal processing and hydrolyses of others CP's. Chemical precipitation is an advantageous process due to its simplicity and low implementation cost. It is a very versatile method that allows the control of product properties such as morphology, size and reactivity, and, therefore, it is a widely used method for the production of nano-particles. Chemical precipitation is normally implemented in stirred reactors, followed by filtering, washing and drying processes (Conn and Jessen, ‘Process for Producing Hydroxyapatite’ U.S. Pat. No. 4,32,4772, 1982), (RUDIN et al., ‘Method for Producing Nano-sized Crystalline Hydroxyapatite’ WO0202461, 2002), (Ying, Ahn and Nakahira, ‘Nanocrystalline apatites and composites, prostheses incorporating them, and method for their production’ US6013591, 2000), (Ahn, ‘Tricalcium phosphates, their composites, implants incorporating them, and method for their production’ US2005031704, 2005). Additionally, the processes can include other stages such as micro-wave radiation treatments and/or aging (Murugan e Seeram, ‘Production of Nano-Sized Hydroxyapatite Particles’ US2005226939, 2005), addition of a solvent (sol-gel method) (Ren and Zhou, ‘Method for synthesizing nano Hydroxyapatite micro powder containing carbonate radical’ CN1587195, 2005), and wet chemically seeding stage immediately followed by drying with spray atomization (Luo, ‘Methods of Synthesizing Hydroxyapatite Powders and Bulk Materials’ US5858318, 1999).

However, the implementation of the wet chemical process in stirred thanks has disadvantages related to the calcium phosphate stoichiometry and the process reproducibility in terms of the some properties of the CP's. The stoichiometry, defined as the molar ratio between Ca²⁺ and PO₄³⁻ ions (Ca/P ratio), is a crucial parameter to produce the desired calcium phosphate, see Table 2. For the case of HAp production, the Ca/P ratio is even determining, since for values lower than 1.67, it can be formed a deficient Hydroxyapatite (DHAp), represented by the chemical formula Ca₁₀₋ₓ(HPO₄)ₓ (PO₄)₆₋ₓ (OH)₂₋ₓ. The reproducibility is an issue for industrial-scale batch production, since different batches have commonly different properties, namely purity, crystal size and morphology, crystallinity degree, and particle size distribution.

The mentioned disadvantages are intrinsic to the stirred reactor technique, which through intensive-energy mixing provides a good macromixing quality due to the recirculation patterns inside the reactor. However, in many real applications, the recirculation patterns lead to the formation of stagnation zones and short-cuts that are hardly overcame. Nonetheless, assuming that these problems can be avoided, stirred reactors cannot ensure a good micromixing quality, which is an essential factor for the CP's purity, crystal size and morphology, crystallinity degree, and particle size distribution. Micromixing defines the mixing phenomena at the molecular scale, which is the scale where the chemical reactions occur, and it is determining for the selectivity of the overall process and for the final product properties. Therefore, for the production of nanomaterials, the process should be controlled at that molecular scale, by ensuring a good micromixing quality.

TABLE 2
Influence of Ca/P rates and Calcium Phosphates
formation conditions and stability

| | | |
|---|---|---|
| Calcium | | |
| Phosphate | Ca/P | Stability and formation conditions |

Formed at room temperature

| | | |
|---|---|---|
| MCPM | 0.5 | Formed at pH < 2 (approximately) |
| DCPD | 1.0 | Formed at 2 < pH < 4, nucleation and fast |
| | | growth up to pH = 6.5 |
| DCPA | 1.0 | Formed at higher temperatures than DCPD but |
| | | slightly stable |
| OCP | 1.33 | Nucleation and fast growth for 6.5 < pH < 8, |
| | | more stable than DCPD or ACP at the same range |
| ACP | 1.5 | Formed in an early stage of the reaction when |
| | | precipitation occurs at high concentrations |
| | | and for 4 < pH < 8, but is spontaneously |
| | | converted in DCPD and OCP |
| HAp | 1.67 | Precipitation occurs at pH > 8, and is the |
| | | calcium phosphate most stable |

Formed at high temperature

| | | |
|---|---|---|
| α-TCP | 1.5 | Formed after heating above 1180° C. followed by |
| | | fast cooling. Less stable than DCPD or OCP |
| β-TCP | 1.5 | Formed after heating up to 1189° C. More stable |
| | | than DCPD or OCP at 6 < pH < 8 |
| TTCP | 2.0 | Formed after heating above 1500° C. Unstable |
| | | in acid solutions |

### DESCRIPTION OF THE INVENTION

The present invention provides a method for wet chemical production of calcium phosphate nanoparticles production by controlling the micromixing quality. Mixing is a crucial parameter of industrial processes and micromixing quality for precipitation processes is critical to define the crystals size and shape, as well as the particles size distribution. Ineffective mixing can lead to non-reproducible processes and low quality products, and therefore, it is often necessary to implement complex purification processes downstream the reactor, increasing the cost of the final product.

Motionless or static mixers have become standard equipment in the process industries since the 1970s, and are employed in a wide range of industries since they couple a better mixing efficiency with lower energy consumption. Static mixers are used inline in an once-through process or in a recycle loop where they supplement or even replace a conventional stirrer. In many continuous processes, static mixers are undeniably an attractive alternative to conventional agitation. Static mixers eliminate the need for mechanical stirrers and therefore have a number of benefits: small space requirement; low equipment cost; no power requirement except pumping; the flow of materials through them may be induced by gravity, pressure difference or by using the existing potential or kinetic energy; easy and quick installation; low set-up and operating costs; self-cleaning; reduced maintenance requirements.

The standard design of a static mixer is a series of identical, flow-modifying motionless inserts, called elements, built into a tubular housing. These motionless inserts use the pressure difference or the kinetic and potential energy of the processed materials, causing splitting, shifting, shearing, rotating, accelerating, decelerating and recombining of materials.

There are more than two hundred commercial static mixer models currently available. Commercial static mixers are available from a number of manufacturers and in different types, shapes and geometries. Commercial static mixers can be divided into five main families: open designs with helices (e.g. Kenics® static mixer); open design with plates (e.g. LPD®, Komax®, Inliner® and HEV® static mixers); corrugated plates (e.g. SMV® static mixer); multilayer designs (e.g. SMX®, SMXL® and SMRX® static mixers); and closed designs with channels (e.g. ISG® static mixer).

Alternatively, network mixers (Lopes, Laranjeira, Dias e Martins, ‘Network Mixer and Related Mixing Process’ WO2005077508, 2005), differ from static mixers as they are not composed by elements that are inserted inside a tubular housing, but promote mixing without mechanical stirring due to its interior geometry constituted by a series of interconnected channels and chambers. The unique available commercial version is the NETmix® technology (registered Portuguese brand), which enables the fluid micromixing control in an optimized and reproducible way, essential factor for the proper control of complex chemical reactions, like nanoparticles production. The NETmix® reactor consists in a network of interconnected chambers and channels creating zones of complete mixing and of complete segregation, which are carefully designed in order to program the mixing intensity and quality, either locally as well along the reactor. This is one of the main advantages present in the NETmix® reactor when compared with other static mixers where the mixing is difficult to control.

The present invention enables the micromixing quality control and ensures the process reproducibility for calcium phosphates nanoparticles produced by wet chemical precipitation implemented in a network mixer or static mixer reactor, fed by a calcium solution, a phosphorous solution, and an alkaline solution; and, optionally, a solvent or a surfactant agent. This process controls the mixing at the molecular level, ensuring a good micromixing quality, essential for the production of particles with nanometric structure and high purity, with controlled crystallinity, particle size and crystal size and morphology. This invention also allows programming the reactants injection scheme with a given distribution at the inlet and along the reactor, performed continuously or varying in time, which enables to programme the pH of the reactant media along the reactor in order to produce the CP's with the desired specifications.

### BRIEF FIGURE DESCRIPTION

FIG. 1: Particle size distribution curves of three hydroxyapatite samples produced in the NETmix® reactor.

FIG. 2: X-ray diffraction spectres of three hydroxyapatite samples produced in the NETmix® reactor.

FIG. 3: Scanning Electron Microscopy image of the example 1 hydroxyapatite sample.

FIG. 4: Scanning Electron Microscopy image of the example 2 hydroxyapatite sample.

FIG. 5: Scanning Electron Microscopy image of the example 3 hydroxyapatite sample.

### DETAILED DESCRIPTION OF THE INVENTION

The most widely method used in CP's production is the wet chemical precipitation reaction, at temperatures below 100° C. The rigorous control of the rnicromixing is crucial for the production of CP's by chemical precipitation in order to get high purity and control the crystal size and morphology, particle size distribution and crystallinity. The processes conventionally can not control the micromixing quality and intensity, and, therefore, a rigorous process control of pH, temperature, stirring and reactants feed flow rate is vital to ensure the product purity. Some of the strategies normally applied to improve chemical homogeneity and stoichiometry of resulting CP's are to promote a slow precipitation and to use very diluted reactants solutions.

Because of the above mentioned reasons, the network mixer or static mixer reactors are promising technologies for the continuous production of CP's nanoparticles, or other substituted CP's nanoparticles by including in their crystalline structures other ions, such as F⁻, Na⁺, K⁺, Mg²⁺ and CO₃²⁻. These reactors operate at low temperatures, making possible the production of MCPM, DCPD, DCPA, OCP, ACP and HAp. The production of α-TCP, β-TCP and TTCP requires further thermal processing at high temperatures, as mentioned at Table 2.

The present invention provides a process that enables the micromixing quality control for the CP's wet chemically synthesis, and allows programming the reactants distribution scheme at the inlet and along the reactor, performed continuously or varying in time, which enables to programme the pH of the reactant media along the reactor in order to produce the CP's with the desired specifications. The reactor also has a thermostatization system which enables an easy temperature control, according to the formulation specification.

The reactor used can be a static mixer or a network mixer with a feed system that allows different configurations of reactants injection schemes achieved by means of valves and flow distributors.

The available reactants are:
1. one source of Ca²⁺ ions;
2. one source of PO₄³⁻ ions;
3. one alkaline agent;
4. one solvent or surfactant agent;
5. water to adjust the concentrations of each of the above aqueous solutions.

Solutions with different concentrations and compositions are prepared by mixing different amounts of the reactants mentioned from 1 to 5. These solutions are specified accordingly to the injection scheme chosen to feed the reactor. The distribution of the reactants can be performed at the reactor inlet or along the reactor, continuously or time variably. The synthesized product can be collected as an aqueous suspension at the reactor's outlet or at any position along the reactor. The obtained suspension can be submitted to further separation processes (decantation, centrifugation, filtration or similar) to increase the solids content in the suspension, which is then washed in order to eliminate all the ions of the remaining solution, and finally can be submitted to a drying process. After drying, the powder product can be milled and/or thermally treated (calcination, sintering).

Therefore, the production of calcium phosphates nanoparticles requires the understanding and the control of the micromixing quality and the appropriated reactants injection scheme with distribution at the reactor's inlet or along the reactor, continuously or time variably. This can be performed by feeding to a network mixer or static mixer reactor several streams containing Ca²⁺ and PO₄³⁻ ions, and an alkaline reactant, with possibility of adding one or more solvents and/or one or more surfactant or tensioactive agents.

Therefore, it is necessary to provide to the reactor the following streams:
a) one Ca²⁺ ions source;
b) one PO₄³⁻ ions source;
c) one alkaline source to adjust the reaction pH;
d) solvents and/or surfactant or tensioactive agents;
e) water to adjust the concentrations of each of the above aqueous solutions;
f) prepare all necessary solutions to feed the reactor with different concentration and compositions, obtained by combining in different proportions the reactants mentioned previously: a), b), c), d) and e);
g) use a network mixer or static mixer reactor that ensures an efficient and homogeneous mixing, equipped with feed distributors at the reactor's inlet and/or along the reactor to allow different reactants injection schemes;
h) the reactant solutions mentioned in f) can be fed at the so said reactor, through a distribution scheme at the inlet or along the reactor, in a continuous or time varying mode;
i) thermostatization of the reactants and/or the reactor in order to ensure a proper temperature for the reaction, where the rigorous choice of operating conditions for the steps defined at f), g), h) and
i) enables the nanoparticles or microparticles production with nanometric crystalline or amorphous structures.

The Ca/P molar ratio should be comprised between the values of 0.5≦Ca/P≦2.0, so that calcium phosphates can be obtained in anhydrous or hydrated forms.

The said calcium phosphates can have some stoichiometric variations resulting from substitution of some ions of the crystallographic structure by other ions, as for example F⁻, Mg²⁺, Na⁺, CO3²⁻ and K⁺.

The said calcium phosphates exhibit nanometric structure (crystal size in the nanometric range) and can be provided as nanoparticles or microparticles.

The said calcium phosphates can have a controlled crystallinity degree, which can vary from amorphous to crystalline structure.

The said calcium phosphates can have a controlled morphology, which can vary from spherical to needle-like geometry.

The calcium phosphates suspension produced in the so said reactor can be further processed to concentration, separation, drying, thermal treatment and/or milling stages to obtain final products in the form of suspensions, slurries or dried powders, with a concentration range varying from 0.1% to 100% of any specific calcium phosphate or a mixture of different calcium phosphates.

The present invention provides a methodology to produce calcium phosphates nanoparticles with high purity, to be applied in several industrial fields, namely in food industry, as food additives and nutritional supplements, in biomaterials as bone graft for bone replacement, growth and repair, biocements and metallic prosthesis coatings. It can also be used as catalysts for water treatment and as absorbents in chromatographic columns. Recent applications include drug delivery, cosmetics, tooth paste and in esthetical treatments to diminishing wrinkles by stimulating conjunctive tissue formation.

### Nanoparticles Characterization

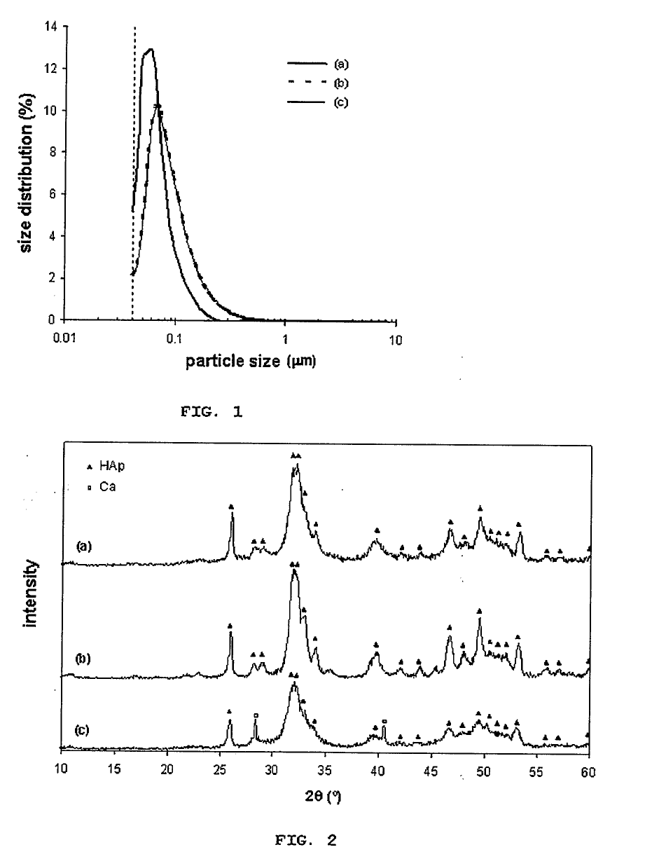

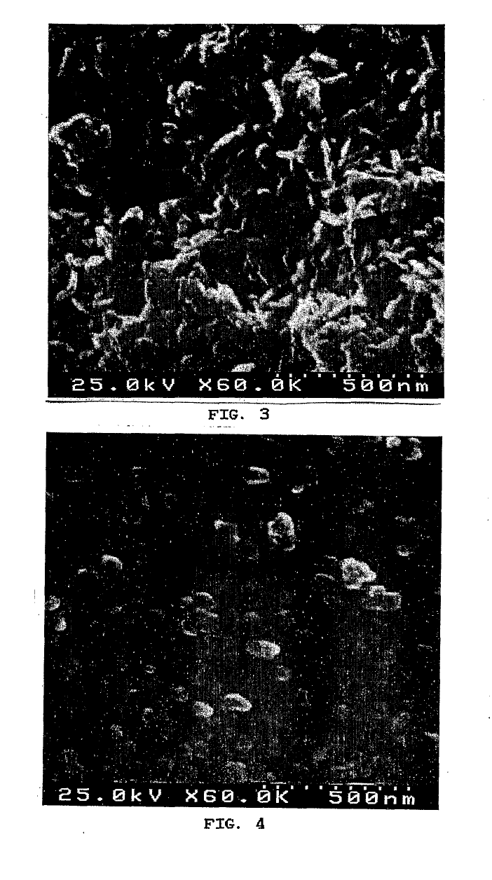

X-Ray Diffractometer (Philips X'pert mod. MPD, Netherland) was used to determine the crystalline phases presented in the nanoparticles produced in the reactor with micromixing quality control. The diffractograms (FIG. 2) were obtained with Cu Ka (λ=1,54056 nm) produced at 40 kV and 50 mA and diffraction angles between 3°<2θ<60° with a step size of 0.05° 2θ/s. The crystallite size was estimated by the Sherrer formula

Laser Diffraction Particle Size Analyzers (Beckman Coulter LS 230, equipped with Polarization Intensity Differential Scattering, USA, Fraunhofer optical model, with shape factor=1.0, i.e., the height/diameter particle ratio is 1:1) was used to determine the particle size distribution curves (FIG. 1) and average particles diameters. The lower detection limit for particles size is of 40 mn.

BET Surface Area Analyser (Micromeretics Gemini II-2370, with sample degasification temperature of 200° C. in 12 h, 5 point analysis and equilibrium time of 50 s) was used to determine specific surface areas using BET method.

Scanning Electron Microscope (Hitachi S-4100, Japan, Vacc=25 kV) was used to characterize the particles morphology. Samples were prepared by fixing the powder in double-side adhesive conductive carbon tape, and then coating it with carbon (FIGS. 3 to 5).

### APPLICATION EXAMPLES

### Example 1

### Needle-Like Shaped Hydroxyapatite Nanoparticles Production.

Production of hydroxyapatite n anoparticles at 25° C. was performed in the commercial NETmix® reactor with 15 inlet ports to feed the reactants solutions and 15 outlets for product recovery. Preparation of 0.5M Ca(NO₃)₂×4H₂O solution, 0.3M NH₄H₂PO₄ solution and 14 solutions of NUOH with different pH values (between 8 and 14). The calcium and phosphorous solutions were fed in one single reactor point (inlet 1). The ammonia solutions were fed on the inlets 2 to 15, with increasing pH order from port 2 to port 15. The hydroxyapatite suspension produced was analysed to determine the particle size distribution curve (FIG. 1.a), where the average particles diameter was dₚ=63 nm. Due to equipment limitations, it was not possible to obtain size distribution ranges lower than 40 nm. Therefore, the real average particle diameter is lower than the referred value (dₚ<63 nm). The suspension obtained was centrifuged, washed, dried at 80° C. under vacuum and milled. The hydroxyapatite powder was analysed by XRD (FIG. 2.a) proving that the as prepared sample is fairly crystalline and that the average crystals size is d_{c}=5.9±3.6 nm, estimated by the Sherrer formula. The SEM image shows that particle morphology is needle-like (FIG. 3) with a shape factor of 5:1. Therefore, particle average diameter is 5 times lower than that obtained by the Granulometer (dₚ<13 nm).

### Example 2

### Spherical Shaped Hydroxyapatite Nanoparticles Production.

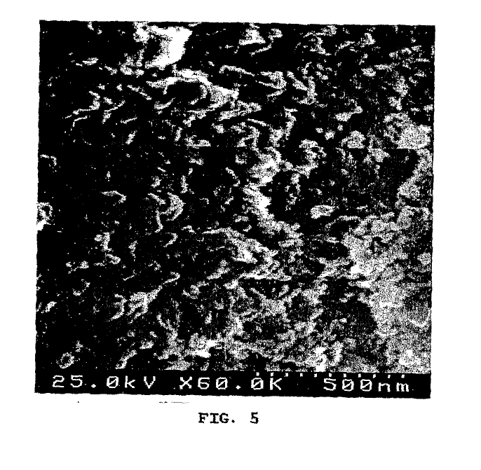

Production of hydroxyapatite n anoparticles at 25° C. was performed in the commercial NETmix® reactor with 15 inlet ports to feed the reactants solutions and 15 outlets for product recovery. Preparation of a Ca(NO₃)₂×4H₂O solution with pH adjusted to 11 by addition of a KOH solution and a NH₄H₂PO₄ solution with pH adjusted to 12 by addition of a KOH solution. Calcium and phosphorous solutions were alternatively fed at the reactor's inlet: odd inlets were used for calcium solution and even inlets for phosphorous solution, guarantying global stoichiometry (Ca/P molar ratio) in any part of the reactor as 10:6. The produced hydroxyapatite suspension was analysed to determine the particle size distribution curve (FIG. 1.b), and the average particles diameter was of dₚ=82 nm. Due to equipment limitations described before, the real average particle diameter is lower than this value. The hydroxyapatite powder was analysed by XRD (FIG. 2.b) proving that the sample is highly crystalline and that the average crystals average size is d_{c}=4.9±1.3 nm. The SEM image shows particles with spherical morphologies (FIG. 4). Therefore, the shape factor used in the Laser Diffraction particle size analysis was very similar to the real value. This sample presented a specific surface BET area of 80.3 m²/g.

### Example 3

Spherical Shaped Hydroxyapatite Nano Particles Production with Low Crystallinity.

Production of hydroxyapatite n anoparticles at 25° C. was performed in the commercial NETmix® reactor with 15 inlet ports to feed the reactants solutions and 15 outlets for product recovery. Preparation of a CaCl₂ aqueous solution containing 20% in volume of ethanol and pH=11 (by adding a KOH solution) and KH₂PO₄ aqueous solution containing 20% in volume of ethanol and pH=12 (by adding a KOH solution). The calcium and phosphorous solutions were fed alternatively at the reactor's inlet: odd inlets were used for calcium solutions and even inlets for phosphorous solution, guarantying the global stoichiometry (Ca/P molar ratio) in any part of the reactor as 10:6. FIG. 1.c shows that hydroxyapatite particles size distribution curve is very similar to one of example 2. However, this sample has lower crystallinity than the previous one, as it can be confirmed by the XRD analysis (FIG. 2.c). The SEM image shows spherical shaped particles morphology (FIG. 5).

1. A method for the production of calcium phosphates particles with high purity comprised by controlling the micromixing quality and distribution injections schemes of reactants at the reactor's inlet or along it, continuously or variably in time.

2. The method according to claim 1, which comprises feeding to a network mixer or static mixer reactor different streams containing Ca²⁺ ions, PO₄³⁻ ions and one alkaline reactant, with possibility of adding one or more solvents and/or one or more surfactant or tensioactive agents.

3. The method according to claim 1, comprised by the following steps:
a) provide one Ca²⁺ ions source;
b) provide one PO₄³⁻ ions source;
c) provide one alkaline source to adjust the reaction pH;
d) provide solvents and/or surfactant or tensioreactive agents;
e) provide water to adjust the concentrations of each of the above aqueous solutions;
f) prepare all necessary solutions to feed the reactor with different concentration and compositions, obtained by combining in different proportions the reactants mentioned previously: a), b), c), d) and e);
g) use a network mixer or static mixer reactor that ensures an efficient and homogeneous mixing, equipped with feed distributors at the reactor's inlet and/or along the reactor to allow different reactants injection schemes;
h) the reactant solutions mentioned in f) can be feed at the so said reactor, through a distribution scheme at the inlet of the reactor or along it, in a continuous or time varying mode;
i) thermostatization of the reactants and/or the reactor in order to ensure a proper temperature for the reaction,
where the rigorous choice of operating conditions for the steps defined at f), g), h) and i) enables the nanoparticles or microparticles production with nanometric crystalline or amorphous structures.

4. The method according to claim 1, wherein the Ca/P molar ratio is in the range 0.5≦Ca/P≦2, so that calcium phosphates can be obtained in anhydrous or hydrated forms.

5. The method according to claim 4, wherein one of the calcium phosphates forms produced is hydroxyapatite.

6. The method according to claim 1, comprised by the calcium phosphates be chemically modified by the substitution of some ions of the crystallographic structure by other ions, as for example F⁻, Na⁺, Mg²⁺ and CO₃²⁻.

7. The method according to claim 1, comprised by the calcium phosphates suspension production in the so said reactor can be further processed to concentration, separation, drying, thermal treatment and/or milling stages to obtain final products in the form of suspensions, slurry or dried powder, with a concentration range varying from 0.1% to 100% of any specific calcium phosphate or a mixture of different calcium phosphates.

8. Nanoparticles and/or microparticles of calcium phosphates having high purity and nanometric structure, comprised by being produced by the method in accordance with claim 1.

9. Nanoparticles and/or microparticles of calcium phosphates having high purity and nanometric structure, according to claim 8, wherein the crystallographic structure has a controlled crystallinity degree, which can vary from amorphous structure to crystalline structure.

10. Nanoparticles and/or microparticles of calcium phosphates having high purity and nanometric structure, according to claim 8, wherein the morphology is controlled, from spherical to needle-like geometry.

11. Use of the nanoparticles and/or microparticles according to claim 8, comprised by production of biomaterials used in biomedicine, preferably as bone graft for bone replacement, growth and repair, biocements and metallic prosthesis coatings.

12. Use of the nanoparticles and/or microparticles according to claim 8, comprised by their application in food industry, preferably as alimentary additives and nutritional supplements.

13. Use of the nanoparticles and/or microparticles according to claim 8, comprised by their application in pharmaceutical industry, preferably in drug delivery and controlled release.

14. Use of the nanoparticles and/or microparticles according to claim 8, comprised by their application in cosmetics industry, preferably in teeth related products and cosmetic products.

15. Use of the nanoparticles and/or microparticles according to claim 8, comprised by their application esthetical treatment to stimulating conjunctive tissue formation.

16. Use of the nanoparticles and/or microparticles according to claim 8, comprised by their application as catalysts for water treatment and as absorbents in chromatographic columns.
